(19) United States
(12) Patent Application Publication
(10) Pub. No.: US 2025/0099913 A1
(43) Pub. Date: Mar. 27, 2025

(54) HUMIDITY CONTROL AGENT AND DEVICE

(71) Applicant: DAIKIN INDUSTRIES, LTD., Osaka (JP)

(72) Inventors: Toshiyuki ITOH, Nagakute-shi (JP); Ryo INOUE, Osaka (JP); Shuji IKEGAMI, Osaka (JP); Tatsuya ANDO, Osaka (JP)

(73) Assignee: DAIKIN INDUSTRIES, LTD., Osaka (JP)

(21) Appl. No.: 18/977,169

(22) Filed: Dec. 11, 2024

Related U.S. Application Data
(63) Continuation of application No. PCT/JP2023/023370, filed on Jun. 23, 2023.

(30) Foreign Application Priority Data
Jun. 24, 2022 (JP) 2022-101625

(51) Int. Cl. B01D 53/28; C07D 207/323; C07F 9/09; F24F 3/14; F25B 37/00

(52) U.S. Cl. CPC B01D 53/28; B01D 2253/20; C07D 207/323; C07F 9/09; F24F 3/1411; F25B 37/00

(57) ABSTRACT

A moisture control material including a salt composed of a pyrazolium cation represented by the formula (N1) and a phosphate ester anion. In formula (N1), R^{N1}, R^{N2}, R^{N3}, R^{N4} and R^{N5} are each independently a hydrogen atom, or an alkyl group having 1 to 12 carbon atoms which optionally has a hydroxy group and optionally includes one or more oxygen atoms in the chain. One or more of R^{N1} and R^{N2} which are substituents on a nitrogen atom that constitutes a 5-membered ring are alkyl groups having 1 to 12 carbon atoms, which optionally have a hydroxy group and optionally include one or more oxygen atoms in the chain.

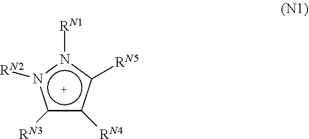

### CROSS-REFERENCE TO RELATED APPLICATIONS

This application is a Rule 53 (b) Continuation of International Application No. PCT/JP2023/023370 filed on Jun. 23, 2023, which claims priority based on Japanese Patent Application No. 2022-101625 filed on Jun. 24, 2022, the respective disclosures of which are incorporated herein by reference in their entirety.

### TECHNICAL FIELD

The present disclosure relates to a moisture control material for use in an air conditioner and an absorption refrigerator, and an apparatus such as an air conditioner and an absorption refrigerator.

### BACKGROUND ART

A desiccant-type air conditioner uses a liquid moisture control material having properties of absorbing water vapor in the air. For example, in Non Patent Documents 1 and 2, liquid moisture control materials made of a lithium chloride aqueous solution, a calcium chloride aqueous solution, or triethylene glycol are disclosed. In Patent Documents 1 to 4 and Non Patent Documents 3 to 7, liquid moisture control materials made of ionic liquid are disclosed, and as the ionic liquid, salts of bromide anions and tetrafluoroborate, dimethyl phosphate anions or methylsulfate anions with imidazolium cations, alkyl phosphonium cations, quaternary ammonium cations or cholinium cations are disclosed. In Non Patent Documents 8 and 9, salts of dimethyl phosphate anions with cholinium cations, dicationic and cyclic quaternary ammonium cations are disclosed.

### RELATED ART

### Patent Document

Patent Document 1: Japanese Translation of PCT International Application Publication No. 2014-505586

Patent Document 2: Japanese Patent Laid-Open No. 2017-221940

Patent Document 3: Japanese Patent Laid-Open No. 2017-154076

Patent Document 4: Japanese patent Laid-Open No. 2016-052614

### Non Patent Documents

Non Patent Document 1: L. Mei, Y. J. Dai, A technical review on use of liquid-desiccant dehumidification for air-conditioning application, Renewable and Sustainable Energy Reviews, 2008, 12, 662-689.

Non Patent Document 2: R. P. Singh, V. K. Mishra, R. K. Das, Desiccant materials for air conditioning applications: A review, lop Conference Series. Materials Science and Engineering, 404 (2018), 012005.

Non Patent Document 3: L. E. Ficke, J. F. Brennecke, Interactions of Ionic Liquids and Water, J. Phy. Chem. B 114 (2010) 10496-10501.

Non Patent Document 4: L. Jing, Z. Danxing, F. Lihua, W. Xianghong, D. Li, Vapor Pressure Measurement of the Ternary Systems H₂O+LiBr+[Dmim]Cl, H₂O+LiBr+[Dmim]BF₄, H₂O+LiCl+[Dmim]Cl, and H₂O+LiCl+[Dmim]BF₄, J. Chem. Eng. Data 56 (2011) 97-101.

Non Patent Document 5: Y. Luo, S. Shao, H. Xu, C. Tian, Dehumidification performance of [EMIM]BF₄, Appl. Thermal Eng. 31 (2011) 2722-2777.

Non Patent Document 6: Y. Luo, S. Shao, F. Qin, C. Tian, H. Yang, Investigation on feasibility of ionic liquids used in solar liquid desiccant air conditioning system, Solar Energy 86 (2012) 2718-2724.

Non Patent Document 7: Watanabe, H.; Komura, T.; Matsumoto, R.; Ito, K.; Nakayama, H.; Nokami, T.; Itoh, T. Design of Ionic Liquids as Liquid Desiccant for an Air Conditioning System, Green Energy & Environment, 4 (2019), 139-145.

Non Patent Document 8: Maekawa, S.; Matsumoto, R.; Ito, K.; Nokami, T.; Li, J-X.; Nakayama, H.; Itoh, T.* Design of Quaternary Ammonium Type-Ionic Liquids as Desiccants for an Air-Conditioning System, Green Chemical Engineering, 1 (2020), 109-116.

Non Patent Document 9: Dicationic Type Quaternary Ammonium Salts as Candidates of Desiccants for an Air-Conditioning System, Itoh, T.; Hiramatsu, M.; Kamada, K.; Nokami, T.; Nakayama, H.; Yagi, K.; Yan, F.; Kim, H-J. ACS Sustainable Chem. Eng., 9 (2021), 14502-14514.

### SUMMARY

The present disclosure provides a moisture control material comprising a salt composed of a pyrazolium cation represented by the following formula (N1) and a phosphate ester anion:

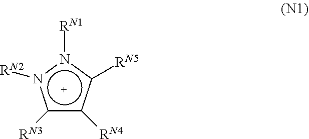

wherein in formula (N1), R^{N1}, R^{N2}, R^{N3}, R^{N4} and R^{N5} are each independently a hydrogen atom, or an alkyl group having 1 to 12 carbon atoms which optionally has a hydroxy group and optionally includes one or more oxygen atoms in the chain; R^{N1}, R^{N2}, R^{N3}, R^{N4} and R^{N5} are the same or different from each other; and one or more of R^{N1} and R^{N2} which are substituents on a nitrogen atom that constitutes a 5-membered ring are alkyl groups having 1 to 12 carbon atoms, which optionally have a hydroxy group and optionally include one or more oxygen atoms in the chain.

### Effects

According to the present disclosure, a moisture control material having high moisture absorption properties can be provided.

### BRIEF DESCRIPTION OF DRAWINGS

FIG. 1 is a graph showing measurement results of the water vapor absorption capacity of a specific salt (8) in Example.

In FIGS. 2A-2D, FIG. 2A is a graph showing measurement test results of the equilibrium water vapor pressure of aqueous solution of 80 mass % specific salt (8) in Example (EtMePyra in FIG. 2D), FIG. 2B is a graph showing measurement test results of the equilibrium water vapor pressure of aqueous solution of 80 mass % specific salt (9) in Example (BuMePyra in FIG. 2D), and FIG. 2C is a graph showing measurement test results of the equilibrium water vapor pressure of aqueous solution of 80 mass % specific salt (10) in Example (OctMePyra in FIG. 2D).

FIG. 3 is a graph showing measurement results of temperature-variable viscosity of aqueous solutions of 70mass % specific salts (8), (9) and (10), and [DETMC6] [DMPO₄]₂ and 1-butyl-3-methylimidazolium=dimethyl phosphate (Bmim-DMPO4) in Comparative Example, respectively.

### DESCRIPTION OF EMBODIMENTS

The lithium chloride aqueous solution and the calcium chloride aqueous solution disclosed in Non Patent Documents 1 to 2 have an advantage of being able to obtain low-humidity air in a stable state. However, in general, aqueous solutions of alkali metal salts and alkaline earth metal salts of these halide ions are corrosive to metals. Therefore, when these substances are applied to a moisture control material, there exists a problem that a highly corrosion-resistant material such as titanium is required for use in a part in contact with the moisture control material in apparatuses such as air conditioners and absorption refrigerators.

The moisture control materials disclosed in Patent Documents 1 to 5 and Non Patent Documents 4 to 5 includes an ionic liquid composed of imidazolium cations and bromide anions and tetrafluoroborate anions as constituent anions. The bromide anions and the tetrafluoroborate anions have problems of difficult handleability in addition to corrosiveness to metals.

Although an ionic liquid composed of cholinium cations and lactic anions having high moisture absorption properties is reported in Patent Document 1, lactic anions are unstable and cannot withstand long-term use. In Non Patent Document 3, it is reported that moisture absorption property of the ionic liquid of imidazolium salt mainly depends on anions, and that acetate anions have good water absorption properties. However, only imidazolium cations have been studied as cations, and an aqueous solution of imidazolium ionic liquid is highly corrosive to metals such as copper (Non Patent Document 7). It is also known that ionic liquids of acetate anions are unstable and cannot withstand long-term use (Non Patent Document 7). In order to solve such problems, in Non Patent Documents 7 to 9, dimethyl phosphate and methyl sulfate are given attention as anions for the selection of ionic liquids. In Non Patent Document 7, it is clarified that an ionic liquid including a combination of phosphonium cations and dimethyl phosphate anions has excellent moisture absorption properties, and in Non Patent Document 8, it is reported that an ionic liquid aqueous solution including a combination of cholinium cations and dimethyl phosphate anions exhibits high moisture absorption properties with low corrosiveness to metals. Further, in Non Patent Document 9, it is reported that quaternary ammonium=dimethyl phosphate salt, in particular, dicationic ammonium, exhibits extremely high moisture absorption properties. It has been found that although dicationic ammonium=dimethyl phosphate exhibits moisture absorption properties superior to all the conventionally reported moisture control materials, the aqueous solution of dicationic ammonium=dimethyl phosphate salt has a relatively high viscosity, (Non Patent Document 9).

In particular, many moisture control materials have been studied as described above, and a novel moisture control material having better moisture absorption properties (moisture absorption capacity and water vapor exchange capacity) is required recently.

The present disclosure has been made to solve the problem, and the object thereof is to provide a moisture control material that exhibits noticeably excellent moisture absorption properties.

Based on the above problems, the present inventors have conducted studies and found that the above problems can be solved by using a moisture control material containing a salt composed of pyrazolium cations and phosphate ester anions, having a specific structure.

Specifically, the problem has been solved by the following means.
<1> A moisture control material comprising a salt composed of a pyrazolium cation represented by the following formula (N1) and a phosphate ester anion:

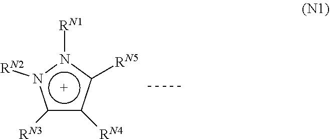

wherein in formula (N1), R^{N1}, R^{N2}, R^{N3}, R^{N4} and R^{N5} are each independently a hydrogen atom, or an alkyl group having 1 to 12 carbon atoms which optionally has a hydroxy group and optionally includes one or more oxygen atoms in the chain; R^{N1}, R^{N2}, R^{N3}, R^{N4} and R^{N5} are the same or different from each other; and one or more of R^{N1} and R^{N2} which are substituents on a nitrogen atom that constitutes a 5-membered ring are alkyl groups having 1 to 12 carbon atoms, which optionally have a hydroxy group and optionally include one or more oxygen atoms in the chain.
<2> The moisture control material according to item <1>, wherein the phosphate ester anion is an anion represented by the following formula (N3):

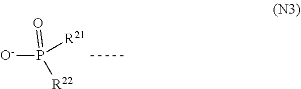

wherein in formula (N3), R²¹ is a hydrogen atom, a hydroxyl group, an alkyl group having 1 to 6 carbon atoms, a hydroxyalkyl group having 1 to 6 carbon atoms, an alkoxy group having 1 to 6 carbon atoms, an alkenyl group having 2 to 6 carbon atoms, a poly (alkyleneoxy) group having 10 or less carbon atoms, an alkylthio group having 1 to 6 carbon atoms, or a poly (alkylene thio) group having 10 or less carbon atoms; and R²² is a hydrogen atom, an alkyl group having 1 to 6 carbon atoms, an alkoxy group having 1 to 6 carbon atoms, an alkenyl group having 2 to 6 carbon atoms, a poly (alkyleneoxy) group having 10 or less carbon atoms, an alkylthio group having 1 to 6 carbon atoms, or a poly (alkylene thio) group having 10 or less carbon atoms.
<3> The moisture control material according to item <1> or <2>, wherein the pyrazolium cation (N1) is represented by any of the following formulas (1) to (4):

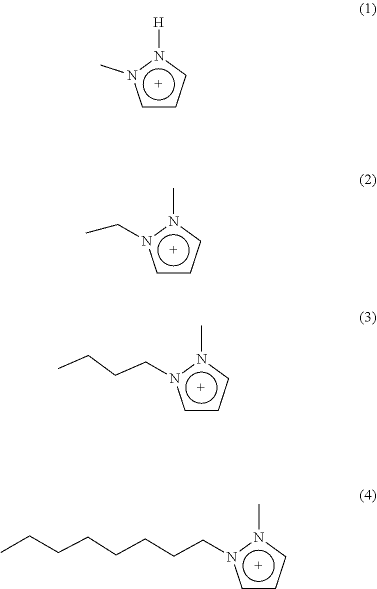

<4> The moisture control material according to any one of items <1>to <3>, wherein the phosphate ester anion is represented by the following formula (17) or (18):

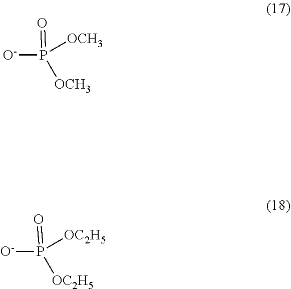 [SMILES: COP(=O)([O-])OC ; CCOP(=O)([O-])OCC]

<5> The moisture control material according to any one of items <1>to <4>, wherein an aqueous solution of the salt composed of the pyrazolium cation represented by the formula (N1) and the phosphate ester anion has a viscosity of 50 mPa·s or less at 25° C.
<6> The moisture control material according to any one of items <1>to <5>, wherein the atmosphere in contact with the aqueous solution of the salt composed of the pyrazolium cation represented by the formula (N1) and the phosphate ester anion has an equilibrium water vapor pressure at 25° C. of 100 hPa or less, and an equilibrium water vapor pressure at 50° C. of 50 hPa or more.
<7> The moisture control material according to any one of items <1>to <6>, wherein the atmosphere in contact with the aqueous solution of the salt composed of the pyrazolium cation represented by the formula (N1) and the phosphate ester anion has a value as difference between an equilibrium water vapor pressure at 25° C. and an equilibrium water vapor pressure at 50° C. (ΔVP₅₀₋₂₅) of 40 hPa or more.
<8> An apparatus comprising the moisture control material according to any one of items <1>to <7>.

An embodiment of the present disclosure is described in detail as follows. Incidentally, the following present embodiment is an example for illustrating the present disclosure, and the present disclosure is not limited to the present embodiment alone.

Incidentally, in the present specification, “to” is used to mean including the numerical values pre- and post-described as a lower limit and an upper limit.

In the present specification, various physical values and characteristic values are ones at 23° C. unless otherwise described.

In the notation of a group (atomic group) in the present specification, a notation without description of substitution or unsubstitution includes a group (atomic group) having no substituent and a group having a substituent (atomic group). For example, “alkyl group” includes not only an alkyl group having no substituent (unsubstituted alkyl group) but also an alkyl group having a substituent (substituted alkyl group). In the present specification, a notation without description of substitution or unsubstitution preferably means unsubstitution.

In the case where a standard shown in the present specification has a different measurement method or the like depending on years, the method or the like is based on the standard as of Jan. 1, 2022 unless otherwise described.

A moisture control material in the present embodiment comprises a salt composed of a pyrazolium cation represented by the following formula (N1) and a phosphate ester anion (hereinafter, described as a specific salt).

By using the specific salt, a moisture control material having excellent moisture absorption properties (moisture absorption capacity and water vapor exchange capacity) can be obtained. Furthermore, the moisture control material in the present embodiment can exhibit a low viscosity. In addition, the moisture control material of the present embodiment can have low corrosiveness to metals.

### <Pyrazolium Cation Represented by Formula (N1)>

The moisture control material of the present disclosure contains a salt composed of a pyrazolium cation represented by the following formula (N1) and a phosphate ester anion.

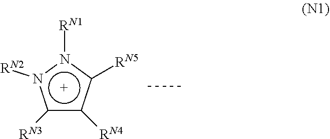

wherein in formula (N1), R^{N1}, R^{N2}, R^{N3}, R^{N4} and R^{N5} are each independently a hydrogen atom, or an alkyl group having 1 to 12 carbon atoms which optionally has a hydroxy group and optionally includes one or more oxygen atoms in the chain; R^{N1}, R^{N2}, R^{N3}, R^{N4} and R^{N5} are the same or different from each other; and one or more of R^{N1} and R^{N2} which are substituents on a nitrogen atom that constitutes a 5-membered ring are alkyl groups having 1 to 12 carbon atoms, which optionally have a hydroxy group and optionally include one or more oxygen atoms in the chain.

In formula (N1), R^{N1}, R^{N2}, R^{N3}, R^{N4} and R^{N5} are each independently a hydrogen atom, or an alkyl group having 1 to 12 carbon atoms which optionally has a hydroxy group and optionally includes one or more oxygen atoms in the chain. In the present embodiment, it is preferable that no hydroxy group be contained. The alkyl group having 1 to 12 carbon atoms has more preferably 1 or more carbon atoms, optionally has 2 or more, 3 or more, 4 or more, 5 or more, 6 or more, or 7 or more carbon atoms, and optionally has 11 or less, 10 or less, 9 or less, 8 or less, 7 or less, 6 or less, 5 or less, or 3 or less carbon atoms. With increase in the number of carbon atoms, the moisture absorption properties tend to be more improved. With decrease in the number of carbon atoms, the viscosity tends to be lowered.

R^{N1}, R^{N2}, R^{N3}, R^{N4} and R^{N5} are optionally the same or different from each other. One or more of R^{N1} and R^{N2} substituted on a nitrogen atom that constitutes a 5-membered ring are alkyl groups having 1 to 12 carbon atoms, which optionally have a hydroxy group and optionally include one or more oxygen atoms in the chain. The number of oxygen atoms that are optionally contained in the alkyl group having 1 to 12 carbon atoms is preferably 0 to 8, more preferably 0 to 4, still more preferably 0 to 2. In the present embodiment, it is preferable that no oxygen atom be contained in the alkyl group having 1 to 12 carbon atoms.

Optionally, R^{N1}, R^{N2}, R^{N3}, R^{N4} and R^{N5} further has a substituent (for example, a hydroxy group, a sulfanyl group (thiol group), etc.) as long as the performance is not greatly impaired. Alternatively, R^{N1}, R^{N2}, R^{N3}, R^{N4} and R^{N5} optionally have a substituent and a specific alkyl group, etc., on the carbon atom of pyrazole scaffold in the range where the performance is not greatly impaired. However, in the present embodiment, it is preferable that no substituent be included.

In an example of R^{N1} and R^{N2} for use in the present embodiment, one of R^{N1} and R^{N2} is a hydrogen atom or an alkyl group having 1 to 5 carbon atoms (preferably 1 to 3 carbon atoms, more preferably 1 to 2 carbon atoms, still more preferably 1 carbon atom), and another is an alkyl group having 1 to 12 carbon atoms (preferably 2 or more carbon atoms, more preferably 3 or more carbon atoms, still more preferably 5 or more carbon atoms, further preferably 6 or more carbon atoms, and preferably 11 or less carbon atoms). With such a composition, the effect of the present disclosure tends to be more effectively exhibited.

In R^{N1} and R^{N2} for use in the present embodiment, the difference between the number of carbon atoms that the alkyl group of R^{N1} has and the number of carbon atoms that the alkyl group of R^{N2} has is preferably 1 or more, more preferably 3 or more, still more preferably 5 or more. The upper limit is, for example, 10 or less. With such a composition, the effect of the present disclosure tends to be more effectively exhibited.

### <Pyrazolium Cation Represented by Formula (1-1)>

The pyrazolium cation represented by the formula (N1) is preferably a pyrazolium cation represented by the following formula (1-1).

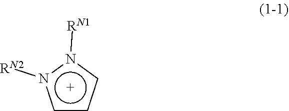

In the formula (1-1), R^{N1} and R^{N2} are the same as in the formula (N1).

It is preferable that the pyrazolium cation for use in the present embodiment have a molecular weight of 110 to 500.

Specific examples (1) to (4) of the pyrazolium cation for use in the present embodiment are shown in the following, though the present disclosure is not limited thereto without mentioning.

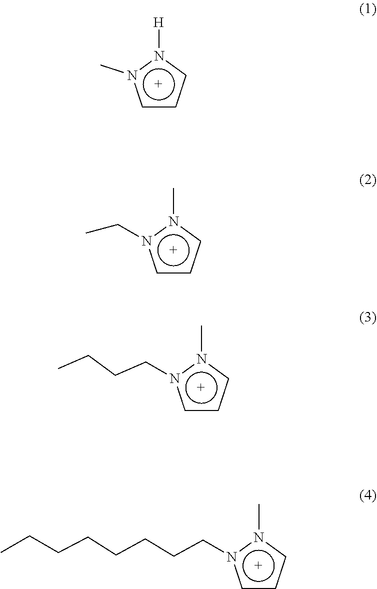

### <Phosphate Ester Anion>

The types and the like of the phosphate ester anion for use in the present embodiment are not particularly specified as long as a salt can be formed with the specific pyrazolium cation, and in particular, a phosphate ester anion represented by a formula (N3) is preferred.

Use of such a phosphate ester anion allows a stable salt to be formed with the pyrazolium cation represented by the formula (N1), so that generation of odor can be effectively suppressed. Incidentally, the term phosphate ester anion in the present disclosure includes, an anion of phosphonate (phosphite) ester and an anion of phosphinate (hypophosphite) ester in addition to an anion of phosphate ester.

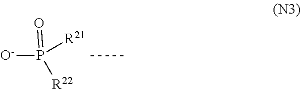

In the formula (N3), R²¹ is a hydrogen atom, a hydroxyl group, an alkyl group having 1 to 6 (preferably 1 to 3) carbon atoms, a hydroxyalkyl group having 1 to 6 (preferably 1 to 3) carbon atoms, an alkoxy group having 1 to 6 (preferably 1 to 3) carbon atoms, an alkenyl group having 2 to 6 (preferably 2 to 3) carbon atoms, a poly (alkyleneoxy) group having 10 or less (preferably 3 to 8) carbon atoms, an alkylthio group having 1 to 6 (preferably 1 to 3) carbon atoms, or a poly (alkylene thio) group having 10 or less (preferably 3 to 8) carbon atoms;

R²² is a hydrogen atom, an alkyl group having 1 to 6 (preferably 1 to 3) carbon atoms, an alkoxy group having 1 to 6 (preferably 1 to 3) carbon atoms, an alkenyl group having 2 to 6 (preferably 2 to 3) carbon atoms, a poly (alkyleneoxy) group having 10 or less (preferably 3 to 8) carbon atoms, an alkylthio group having 1 to 6 (preferably 1 to 3) carbon atoms, or a poly (alkylene thio) group having 10 or less (preferably 3 to 8) carbon atoms.

In particular, R²¹ and R²² are preferably an alkyl group having 1 to 6 (preferably 1 to 3) carbon atoms and an alkoxy group having 1 to 6 (preferably 1 to 3) carbon atoms, more preferably an alkoxy group having 1 to 6 (preferably 1 to 3) carbon atoms, still more preferably alkoxy group having 1 to 3 carbon atoms, and further preferably a methoxy group and an ethoxy group.

It is preferable that the phosphate ester for use in the present embodiment have a molecular weight of 64 to 650.

Specific examples of the phosphate ester anion for use in the present embodiment are shown in the following, with proviso that the phosphate ester anions in the present embodiment are not limited thereto without mentioning.

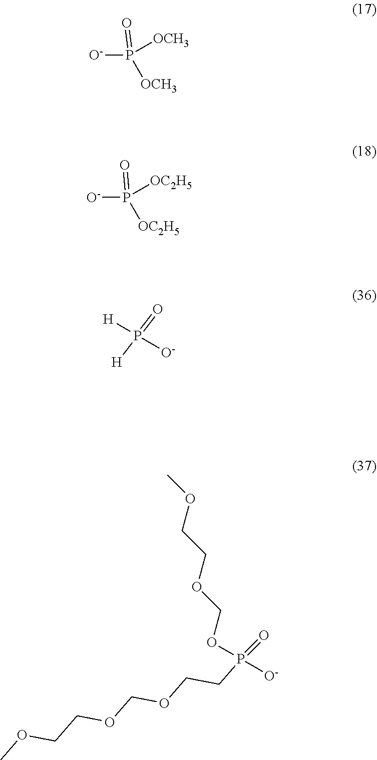

### <Pyrazolium Cationic Salt>

Specific examples of the salt of pyrazolium=alkyl phosphate as specific salt include a salt represented by the following formula (N4), more specifically a salt represented by the following formulas (7), (8), (9) and (10).

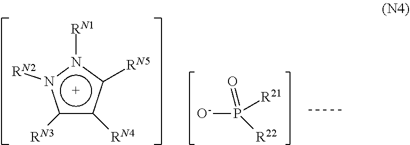

R^{N1} to R^{N5} in the formula (N4) are the same as in the formula (N1). R²¹ and R²² in the formula (N4) are the same groups as in the formula (N3).

For example, typical examples of the salt composed of the pyrazolium cation represented by the formula (1-1) and a dimethyl phosphate anion include the following chemical formulas (7) to (10).

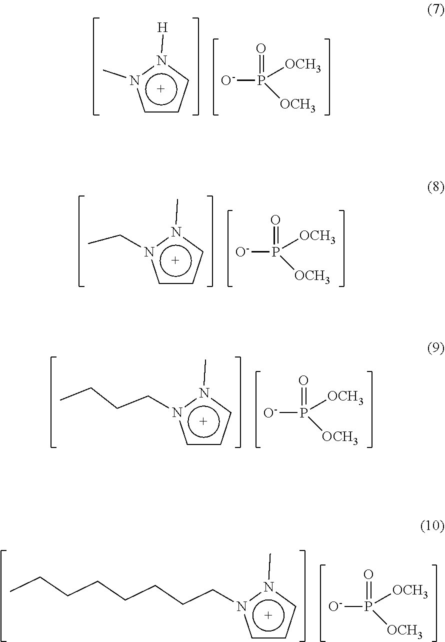

A pyrazole having a structure of the pyrazolium cation described in formula (N1) with any one of R^{N1} and R^{N2} excluded is reacted with a trialkyl phosphate having any one of alkoxy groups of R^{N1} and R^{N2} to make an onium compound, so that a specific salt can be synthesized. The synthesizing process allows the reaction to proceed easily, so that a specific salt can be synthesized in a very simple manner. Further, a metal exchange reaction or an anion exchange with use of an ion exchange resin is not required, so that contamination with a halogen that causes corrosion can be suppressed.

Incidentally, the specific salt may be a solid at normal temperature, or may be a liquid (so-called normal-temperature molten salt (ionic liquid)).

### <Moisture Control Material>

The moisture control material may contain one type of specific salt, or may contain two or more specific salts with one or both of the cation and the anion being different.

The moisture control material may further contain other components usually used in moisture control materials in the range where the effect of the present embodiment is not impaired.

The moisture control material may be an aqueous solution. In this case, the specific salt is contained in the cationic and anionic states.

The moisture control material in the present embodiment may be applied to, for example, a desiccant-type air conditioner and an absorption refrigerator. The moisture control material in the present embodiment may be used in any aspect of an open system and a closed system. In the case of use in an aspect of an open system, the moisture control material including the phosphate ester anion that constitutes the specific salt is particularly suitable from the view point of suppression of odor.

In another embodiment, the salt is preferably an ionic liquid. The ionic liquid means one having a melting point of 100° C. or less at one atmospheric pressure. In particular, the moisture control material in the present embodiment is preferably in a liquid state at least in the range of 0 to 5° C.

The moisture control material in the present embodiment may be an aqueous solution as described above, and is preferably an aqueous solution. In this case, as described above, the specific salt is contained in the cationic and anionic states. The amount of water is preferably 10 mass % or more, more preferably 15 mass % or more, relative to the total amount of the salt (ionic liquid) and water. Also, the amount of water is preferably 95 mass % or less, more preferably 80 mass % or less, and particularly preferably 60 mass % or less.

Further, the moisture control material in the present embodiment may further contain other components usually used in moisture control materials in the range where the effect of the present disclosure is not impaired.

The moisture control material in the present embodiment may be used for an apparatus such as an air conditioner and an absorption refrigerator. As examples of such devices, the descriptions in Japanese Patent Laid-Open No. 2006-142121, Japanese Patent Laid-Open No. 2018-144029, Japanese Patent Laid-Open No. 2020-30004, etc. may be referred to. In particular, since the generation of odor is suppressed, use in open systems such as a desiccant-type air conditioner is particularly suitable.

The moisture control material in the present embodiment may be suitably applied to, for example, a desiccant-type air conditioner and an absorption refrigerator. The moisture control material in the present embodiment may be used in any aspect of an open system and a closed system. In the case of use in an aspect of an open system, the moisture control material including the phosphate ester anion that constitutes the specific salt is particularly suitable from the view point of suppression of odor.

The pyrazolium phosphate solution that constitutes the moisture control material in the present embodiment preferably has low viscosity. The viscosity of the aqueous solution (for example, 70 mass % aqueous solution) at 25° C. is preferably 50 mPa·s or less, more preferably 40 mPa·s or less, still more preferably 30 mPa·s or less, further preferably 25 mPa·s or less, and furthermore preferably 20 mPa·s or less. With a low viscosity of the pyrazolium phosphate, it is expected that the transfer efficiency of the moisture control material aqueous solution and the water vapor exchange efficiency resulting from contact with ambient air can be improved. The lower limit is, for example, 5 mPa·s or more, at 25° C.

The aqueous solution in the present embodiment (for example, 70 mass % aqueous solution) has difference between viscosity at 20° C. and viscosity at 50° C. of preferably 40 mPa·s or less, more preferably 20 mPa·s or less. Decrease in the chain length of the alkyl group of the pyrazolium cation allows to reduce the difference in the viscosity at 20° C. and at 50° C.

The lower limit of the difference in the viscosity is preferably more than 0 mPa·s.

The moisture control material in the present embodiment has low corrosiveness to various metals such as zinc, copper, aluminum and stainless steel. In the present embodiment, due to use of a pyrazolium salt, the acidity of hydrogen positioned between N atoms is low, so that metal corrosion tends to be more effectively suppressed.

According to the constitution, a moisture control material having a high moisture absorption coefficient can be obtained. Further, since the generation of odor can be suppressed, use in open systems such as a desiccant-type air conditioner is particularly suitable.

### EXAMPLES

The present disclosure will be described more specifically with reference to Examples as follows. The materials, amounts used, ratios, processing details, processing procedures, etc. shown in the following Examples may be changed appropriately without departing from the gist of the present disclosure. Accordingly, the scope of the present disclosure is not limited to the following specific examples. In the following structural formulas, Et is an ethyl group and Me is a methyl group.

In the case where the measuring instruments and the like used in Examples are hardly available due to discontinued production or the like, other instruments having equivalent performance may be used for the measurement.

Unless otherwise specified, in the present Example, the environment was at 23° C. and a relative humidity of 40%.

### <Synthesis of Specific Salt of Pyrazolium>

### Example 1

### Synthesis of 1-ethyl-2-methylpyrazolium=dimethyl phosphate (8)

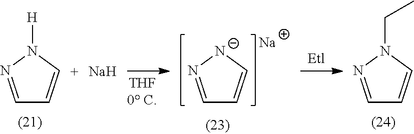

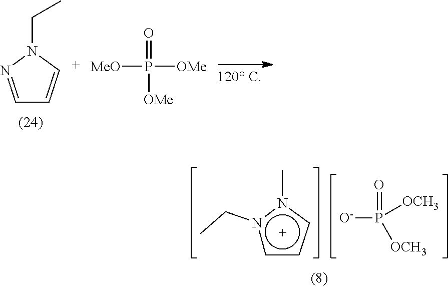

In a 500-mL three-neck eggplant flask equipped with a Dimroth condenser and a 100-mL cylindrical dropping funnel, sodium hydride (NaH) (12.4 g, ca. 60% in mineral oil, 310 mmol) was weighed out, and after purging with argon, dry hexane was added at room temperature to remove supernatant (2 times), so that mineral oil components were removed. Subsequently, 100 mL of dry tetrahydrofuran (THF) was added thereto at room temperature to make a suspension. The flask was immersed in an ice bath, and a dry THE solution (100 mL) of pyrazole (21) (20.42 g, 300 mL) was dropped therein at 0° C. On this occasion, hydrogen gas was generated. While releasing the generated hydrogen gas, the dropping was performed for 1 hour, so that a sodium salt (23) was formed. After completion of the dropping, stirring was performed at room temperature for 1 hour, and then a dry THE solution (100 mL) of ethyl iodide (51.47 g, 330 mmol) was dropped at room temperature over 30 minutes. After completion of the dropping, reflux was performed at 70° C. for 19 hours. After cooling, 5.58 g (310 mmol) of ice pieces were added thereto and stirred at room temperature for 30 minutes. Precipitated sodium iodide was removed through celite filtration, and the content was transferred into a 500-mL one-neck eggplant flask and was concentrated under reduced pressure with a rotary evaporator. After THE as solvent was mostly evaporated, a viglue (vigreux) tube was mounted to perform Claisen distillation under a reduced pressure, so that 1-ethylpyrazole (24) (bp: 72 to 75° C./22 hPa, 21.17 g, 220 mmol,) was obtained at a yield of 73%.

¹H NMR (500 MHz, ppm, CDCl₃) δ 1.47 (3H, t, J=7.2 Hz), 4.17 (2H, q, J=7.2 Hz), 6.22 (1H, t, J=2.4 Hz), 7.37 (1H, d, J=4.0 Hz), 7.48 (1H, d, J=2.4 Hz); ¹³C NMR (20 MHz, ppm, CDCl₃) δ 15.6, 45.8, 105.2, 128.1, 139.0 ppm.

In a 200-mL two-neck eggplant flask equipped with a Dimroth condenser, 1-ethylpyrazole (24) (20.60 g, 214 mmol) was weighed out, and trimethyl phosphate (33.1 g, 236 mmol) was added at room temperature from a side pipe. The inside of the reaction system was replaced with argon gas by flushing with argon gas, and then stirred at 120° C. for 72 hours. After cooling to room temperature, 100 mL of pure water was added therein to make an aqueous solution. The water layer was washed with ether 3 times, freeze dried, and subsequently, dried at 50° C. and 7.1 hPa, for 7 hours, so that pyrazolium salt (8) (49.29 g, 209 mmol) as semi-melted colorless oil component was obtained at a yield of 97%.

¹H NMR (500 MHz, ppm, CDCl₃) δ 1.60 (3H, t, J=7.2 Hz), 3.56 (3H, s), 3.71 (3H, s), 4.42 (2H, q, J=7.2 Hz), 6.80 (1H, t, J=2.4 Hz), 8.16 (2H, t, J=3.2 Hz); ¹³C NMR ((20 MHz, ppm, CDCl₃) δ 13.1, 36.5, 45.3, 53.0, 53.2, 107.3, 137.1, 139.6 ppm. HRMS (ESI) C₆H₁₁N₂⁺111.09227; found 111.0922; HRMS (ESI) calcd for C₂H₆O₄P³¹ 125.000382; found 124.9995.

The structure and chemical formula numbers of the pyrazolium salt as specific salt synthesized by the same method are represented by the specific salts (9) and (10).

### Example 2

### Synthesis of 1-methylpyrazolium=dimethyl phosphate (7)

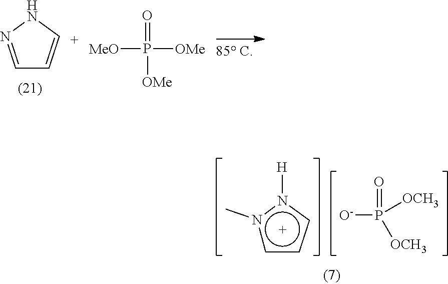

To pyrazole (21) (0.68 g, 10.0 mmol), trimethyl phosphate ester (1.54 g, 11 mmol) was added at room temperature to cause a reaction in an argon gas atmosphere, at 85° C. for 24 hours. After the reaction, the reaction system was cooled to room temperature, and the residue was washed with hexane and diethyl ether and then dried under reduced pressure for 3 hours with a rotary evaporator and vacuum pump at 55° C. (8.5 hPa), so that a specific salt (7) as colorless oil component was obtained at a yield of 81% (1.69 g, 8.12 mmol). The aqueous solution of the specific salt (7) exhibited acidity (pH 3.4), and only moisture absorption capacity was examined. Incidentally, even with addition of trimethyl phosphate ester in amount of 2 equivalents or more, no dimethyl pyrazolium salt was formed.

¹H NMR (80 MHz, ppm, CDCl₃) δ 3.57 (3H, s), 3.76 (3H, s), 4.33 (3H, s), 6.73 (1H, t, J=3.2 Hz), 8.47 (1H, d, J=2.4 Hz), 10.55 (2H, s); ¹³C NMR ((20 MHz, ppm, CDCl₃) δ 37.0, 53.1, 53.3, 107.3, 138.3 ppm.

### <Moisture Absorption Test, Example 3>

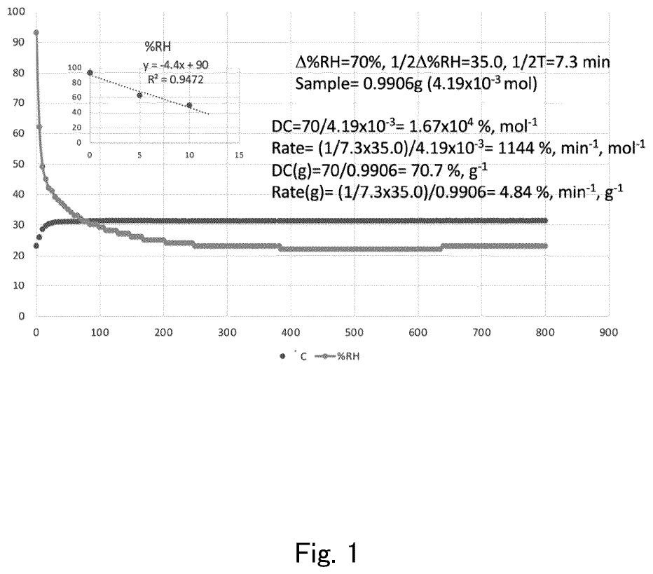

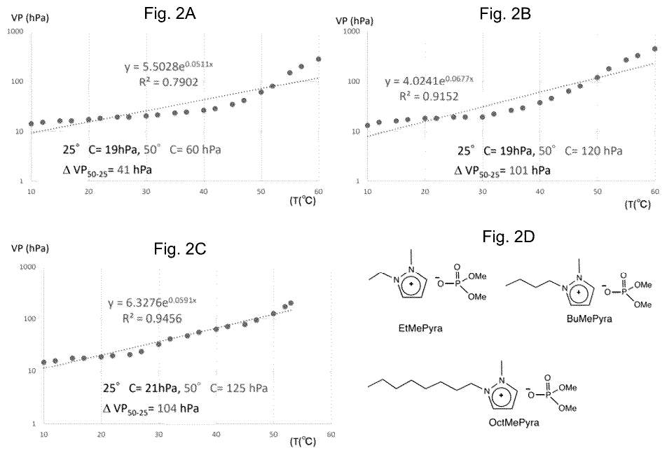

A petri dish with 1.0038 g of the specific salt (8) in Example 1 dispensed therein was placed in a zippered plastic bag having an internal capacity of 1110 cm² (manufactured by Asahi Kasei Home Products Corporation, Ziploc (registered trademark), 273 mm by 268 mm) together with a hygrometer (TR-74Ui, illuminance/ultraviolet/temperature/humidity data logger manufactured by T&D Corporation), and the plastic bag was sealed. This was placed in a constant-temperature vessel at 30° C. and allowed to stand still. The change in humidity inside the plastic bag was measured until the humidity inside the plastic bag reached an equilibrium state. The results are shown in graph in FIG. 1. As shown in FIG. 1, the humidity decreases over time, indicating that the specific salt (8) has moisture absorption properties.

In the same method, 1.000 g of each of the specific salts (7), (9) and (10) was placed in a petri dish and subjected to the same moisture absorption test as for the specific salt (8). The results are shown in Table 1.

Comparison of moisture absorption performance of [DETMC6] [DMPO₄]₂ described in Non Patent Document 9, which exhibited the conventionally best moisture absorption performance, and calcium chloride, which is currently used as dehumidifying agent.

TABLE 1

| | | Moisture | | Moisture | |
|---|---|---|---|---|---|
| | | absorption | Moisture | absorption | Moisture |
| | | capacity | absorption rate | coefficient | absorption rate |
| | Moisture control | per mole | per mole | per gram | per gram |
| | material | (%/mol) | (%/min, mol) | (%/g) | (%/min, g) |
| Example 4-1 | Specific salt (7) | 11400 | 848 | 57 | 4.1 |
| Example 4-2 | Specific salt (8) | 16700 | 1144 | 71 | 4.8 |
| Example 4-3 | Specific salt (9) | 21600 | 1460 | 82 | 5.5 |
| Example 4-4 | Specific salt (10) | 26100 | 809 | 84 | 2.6 |
| Comparative | MOA0090 ^{[b]} | 2300 | 170 | 21 | 1.5 |
| Example 4-1 ^{[a]} | | | | | |
| Comparative | CTH2255 ^{[c]} | 1710 | 687 | 28 | 1.6 |
| Example 4-2 ^{[a]} | | | | | |
| Comparative | [DETMC6] [DMPO₄]₂ | 20000 | 760 | 46 | 1.4 |
| Example 4-3 ^{[a]} | | | | | |
| Comparative | Bmim-DMPO₄ | 7420 | 317 | 28 | 1.2 |
| Example 4-4 | | | | | |

^{[a]} described in Non Patent Document 9
^{[b]} Calcium chloride (NACALAI, Lot No. MOA0090)
^{[c]} Silica gel (FUJIFILM Wako, Lot No. CTH2255)

The specific salts (7) to (10) exhibited a much higher moisture absorption coefficient in comparison with calcium chloride for drying in Comparative Example 4-1 and silica gel for drying in Comparative Example 4-2 shown in Table 1, which are currently widely used as desiccants. Further, the specific salts (7) to (10) exhibited a much higher moisture absorption properties in comparison to Bmim-DMPO₄ (Comparative Example 4-4) as typical ionic liquid. Even the specific salt (7), which has the lowest moisture absorption performance among the specific salts synthesized in this time, exhibited 1.5 times (DC (mol)) per mole higher moisture absorption performance, and the specific salt (10), which has the highest moisture absorption properties among the pyrazolium salts of monocation, exhibited 3.5 times (DC (mol)) per mole higher moisture absorption performance in comparison to Bmim-DMPO₄. The moisture absorption properties per mole of the specific salts (7) to (10) were in the order of (10)>(9)>(8)>(7), and the moisture absorption properties per gram were in the order of (10)>(9)>(8)>(7).

[DETMC6] [DEPO₄]₂ is a compound having the highest level of moisture absorption properties in the world at the time of its paper publication, so that it is presumed that the moisture absorption performance of the specific salt (10) is superior to all the moisture control materials that have been conventionally reported.

Although there has been reported that an ionic liquid having a smaller cation portion has higher moisture absorption properties (Non Patent Document 3), it has been found that the rule does not apply to the specific salts (7) to (10) as ionic liquids of pyrazolium salt.

### <Measurement of Equilibrium Water Vapor Pressure, Example 4>

Aqueous solutions of 80 mass % specific salt (8), (9), and (10) synthesized in Example 1 were prepared to measure the equilibrium water vapor pressure at each temperature ranging from 20° C. to 60° C. The results are shown in FIGS. 2A-2C.

Aqueous solutions of 80 mass % specific salts (8), (9) and (10) and [DETMC6] [DEPO₄]₂, and an aqueous solution of 30 mass % lithium chloride were prepared by the same method to measure the equilibrium water vapor pressure at 25° C. and 50° C. The equilibrium water vapor pressures are shown in FIGS. 2A-2C.

Equilibrium water vapor pressures of the specific salts (8) to (10), [DETMC6] [DEPO₄]₂, and lithium chloride at 25° C. and 50° C.

TABLE 2

| | | Concentration of | Equilibrium water | Equilibrium water |
|---|---|---|---|---|
| | | aqueous solution | vapor pressure | vapor pressure |
| | Specific salt | (mass %) | (25° C.) | (50° C.) |
| Example 5-1 | Specific salt (8) | 80 | 19 hPa | 60 hPa |
| Example 5-2 | Specific salt (9) | 80 | 19 hPa | 120 hPa |
| Example 5-3 | Specific salt (10) | 80 | 21 hPa | 125 hPa |
| Comparative | [DETMC6] [DMP0₄]₂ | 80 | 23 hPa | 140 hPa |
| Example 5-1 ^{[a]} | | | | |
| Comparative | Lithium Chloride | 30 | 24 hPa | 150 hPa |
| Example 5-2 ^{[a]} | | | | |

^{[a]} described in Non Patent Document 9

As shown in Example 5-1 to Example 5-3 in Table 2, the aqueous solutions of 80 mass % specific salts (8) to (10) had a very low equilibrium water vapor pressure of 19 hPa or 21 hPa at 25° C., and it has been found that the equilibrium water vapor pressures are somewhat lower in comparison with the aqueous solution of 30 mass % lithium chloride shown in Comparative Example 5-2 in Table 2.

Since a moisture control material for a liquid-type desiccant air conditioner is required to smoothly absorb and desorb water vapor from and to the ambient air to be contacted, it is desired to have a low equilibrium water vapor pressure at low temperature and a high equilibrium water vapor pressure at high temperature. From such a viewpoint, the equilibrium water vapor pressure at 25° C. is preferably 100 hPa or less, more preferably 70 hPa or less, still more preferably 50 hPa or less, and furthermore preferably 20 hPa or less. The lower limit thereof is practically 5 hPa or more, though not limited. The equilibrium water vapor pressure at 50° C. is preferably 50 hPa or more, more preferably 80 hPa or more, and particularly preferably 100 hPa or more. The upper limit thereof is practically 800 hPa or less, though not limited.

The value as difference between the equilibrium water vapor pressure at 50° C. and the equilibrium water vapor pressure at 25° C. (ΔVP₅₀₋₂₅) is preferably 20 hPa or more, more preferably 30 hPa or more, still more preferably 40 hPa or more, further preferably 75 hPa or more, and furthermore preferably 100 hpa or more. The upper limit of ΔVP₅₀₋₂₅ is practically 200 hPa or less, though not limited.

The results shown in Example 5-1 in Table 2 show that the features of the aqueous solution of 80 mass % specific salt (8) as moisture control material are equivalent to those of the aqueous solution of 30 mass % lithium chloride, which allows smooth water vapor exchange through contact with an ambient air to be achieved.

From the experimental results shown in Examples 5-2 and 5-3 in Table 2, it has been shown that the features of the aqueous solutions of 80 mass % specific salt (9) and specific salt (10) as moisture control material are more excellent than those of an aqueous solution of 30 mass % lithium chloride at low temperature. However, the equilibrium water pressure at 50° C. is low, and in water vapor exchange through contact with an ambient air, the equilibrium water pressure is lower in comparison with 30 mass % lithium chloride. Accordingly, it has been found that the performance of water vapor exchange is low.

### <Viscosity Test, Example 5>

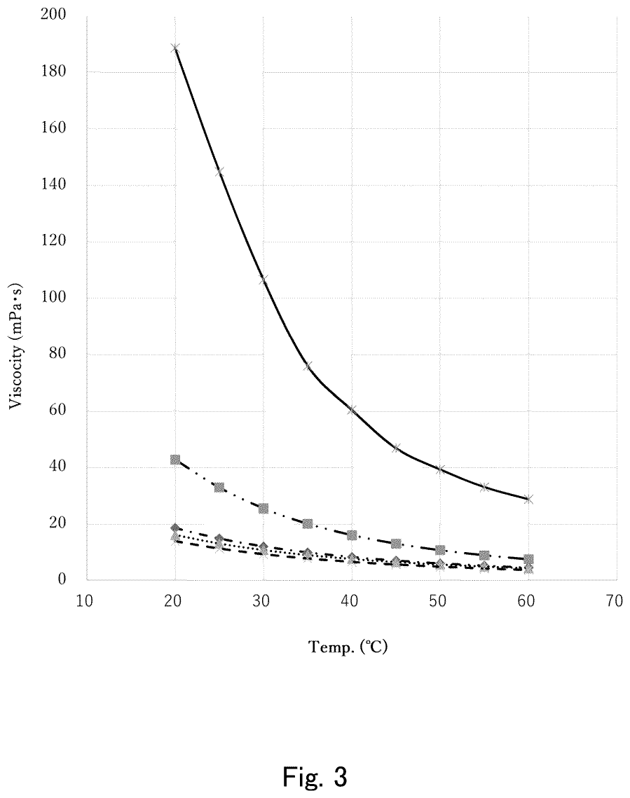

It is desirable that the viscosity of the moisture control material for a liquid-type desiccant air conditioner be as low as possible. As shown in FIG. 3, it has been found that the specific salts of the present disclosure had lower viscosity. In FIG. 3, [DETMC6] [DMPO₄]₂, specific salt (10), specific salt (9), specific salt (8) and Bmim-DMPO₄ are shown in descending order of the viscosity at a temperature (Temp.) of 20° C.

As shown in FIG. 3, it has been found that among the aqueous solutions of 70 mass % specific salt (8) to specific salt (10), the viscosity of the specific salt (10) is high, while Bmim-DMPO₄ has low moisture absorption performance (Comparative Example 4-4 in [Table 1]), with a mole ratio of about 40% of the specific salt (8). It has been also found that at any temperature, the aqueous solutions of 70 masso specific salt (8) to specific salt (10) had a much lower viscosity in comparison with the 70 mass % [DETMC6] [DMPO₄]₂ (Comparative Example 4-3 in [Table 1]). In a liquid-type desiccant air conditioner, the ambient air is brought into contact with the aqueous solution of moisture control material to perform moisture control of the ambient air and air conditioning temperature control. Accordingly, in order to reduce the resistance of the aqueous solution of moisture control material in the piping and to improve the efficiency of contact with the ambient air, it is preferable that the viscosity of the aqueous solution of moisture control material be low. The aqueous solutions of 70 mass % specific salt (8) to specific salt (10) have low viscosity at each temperature, so that efficient moisture control can be achieved through contact with the ambient air.

Incidentally, the measurement of viscosity was performed according to ISO 23581:2020.

1. A moisture control material comprising a salt composed of a pyrazolium cation represented by the following formula (N1) and a phosphate ester anion: 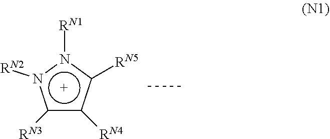
wherein in the formula (N1), R^{N1}, R^{N2}, R^{N3}, R^{N4} and R^{N5} are each independently a hydrogen atom, or an alkyl group having 1 to 12 carbon atoms which optionally has a hydroxy group and optionally includes one or more oxygen atoms in a chain; R^{N1}, R^{N2}, R^{N3}, R^{N4} and R^{N5} are the same or different from each other; and one or more of R^{N1} and R^{N2} which are substituents on a nitrogen atom that constitutes a 5-membered ring are alkyl groups having 1 to 12 carbon atoms, which optionally have a hydroxy group and optionally include one or more oxygen atoms in a chain.

2. The moisture control material according to claim 1, wherein the phosphate ester anion is an anion represented by the following formula (N3): 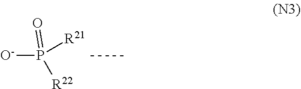
wherein in the formula (N3), R²¹ is a hydrogen atom, a hydroxyl group, an alkyl group having 1 to 6 carbon atoms, a hydroxyalkyl group having 1 to 6 carbon atoms, an alkoxy group having 1 to 6 carbon atoms, an alkenyl group having 2 to 6 carbon atoms, a poly (alkyleneoxy) group having 10 or less carbon atoms, an alkylthio group having 1 to 6 carbon atoms, or a poly (alkylene thio) group having 10 or less carbon atoms; and R²² is a hydrogen atom, an alkyl group having 1 to 6 carbon atoms, an alkoxy group having 1 to 6 carbon atoms, an alkenyl group having 2 to 6 carbon atoms, a poly (alkyleneoxy) group having 10 or less carbon atoms, an alkylthio group having 1 to 6 carbon atoms, or a poly (alkylene thio) group having 10 or less carbon atoms.

3. The moisture control material according to claim 1, wherein the pyrazolium cation (N1) is represented by any of the following formulas (1) to (4):

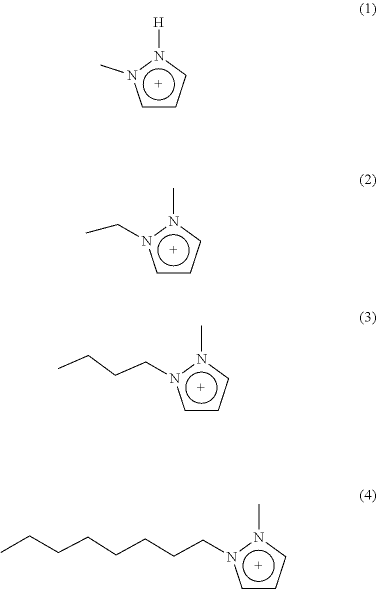

4. The moisture control material according to claim 1, wherein the phosphate ester anion is represented by the following formula (17) or (18):

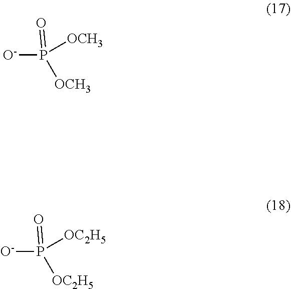 [SMILES: COP(=O)([O-])OC ; CCOP(=O)([O-])OCC]

5. The moisture control material according to claim 1, wherein an aqueous solution of the salt composed of the pyrazolium cation represented by the formula (N1) and the phosphate ester anion has a viscosity of 50 mPa·s or less at 25° C.

6. The moisture control material according to claim 1, wherein the atmosphere in contact with the aqueous solution of the salt composed of the pyrazolium cation represented by the formula (N1) and the phosphate ester anion has an equilibrium water vapor pressure at 25° C. of 100 hPa or less, and an equilibrium water vapor pressure at 50° C. of 50 hPa or more.

7. The moisture control material according to claim 1, wherein the atmosphere in contact with the aqueous solution of the salt composed of the pyrazolium cation represented by the formula (N1) and the phosphate ester anion has a value as difference between an equilibrium water vapor pressure at 25° C. and an equilibrium water vapor pressure at 50° C. (ΔVP₅₀₋₂₅) of 40 hPa or more.

8. An apparatus comprising the moisture control material according to claim 1.
